(19) United States
(12) Patent Application Publication
(10) Pub. No.: US 2002/0024020 A1
(43) Pub. Date: Feb. 28, 2002

(54) Electron-beam drawing appraratus electron-beam drawing method

(76) Inventors: Kimiaki Ando, Hamura (JP); Haruo Yoda, Hinode-machi (JP); Rikio Tomiyoshi, Mito (JP); Masamichi Kawano, Mito (JP)

Correspondence Address: KENYON & KENYON, ONE BROADWAY, NEW YORK, NY, 10004, US

(21) Appl. No.: 09/935,185

(22) Filed: Aug. 22, 2001

(30) Foreign Application Priority Data
Aug. 29, 2000 (JP) 2000-259478

(51) Int. Cl. G21G 5/00; A61N 5/00

(52) U.S. Cl. 250/492.3

(57) ABSTRACT

In the case of drawing an oblique figure pattern, when drawing an oblique figure by using a slender rectangular beam, a problem occurs that edge roughness occurs at an oblique-side portion to deteriorate the drawing accuracy. The present invention solves the above problem and provides an electron-beam drawing apparatus and an electron-beam drawing method capable of accurately drawing even an oblique figure.

A first rectangular aperture and a second parallelogrammatic aperture are used and a variable parallelogrammatic electron beam formed by two apertures is used to draw a desired pattern on the surface of a sample. Moreover, oblique-side-portion-contour decomposition means is used to draw an oblique-side portion by a variable parallelogram and the inside of an oblique side by a triangle and a quadrangle (rectangle).

### BACKGROUND OF THE INVENTION

The present invention relates to an electron-beam drawing apparatus and an electron-beam drawing method for drawing a minute pattern by using an electron beam, particularly to improvement of the oblique-side drawing accuracy of an oblique figure.

A conventional electron-beam drawing apparatus draws a pattern by storing an LSI pattern to be drawn in a drawing-pattern memory, decomposing the LSI pattern into a minute rectangular pattern which can be drawn by a figure decomposition circuit or the like, and applying an electron beam formed by a plurality of apertures onto a wafer.

FIG. 12 is an illustration showing a conventional drawing method using a rectangular aperture. An electron beam 101 passes through a first aperture having a rectangular opening 102 to form a rectangular beam 110 through mutual cutting (beam passing through the opening of its own) with a second aperture having a second rectangular opening 106. The beam passing through the first aperture generates a variable rectangular beam by controlling a beam passing through the second aperture deflected by a deflecting system 120.

FIGS. 13A to 13E are illustrations for explaining deterioration of the accuracy of a minute pattern, particularly an oblique-side figure formed by a conventional rectangular beam. As shown in FIG. 13A, a triangular FIG. 701 has been drawn by using a variable rectangle 702. Because a drawn pattern is recently further fined and the height-directional size of the rectangle 702 is large, a problem occurs that an oblique-side portion 703 becomes stepwise and edge roughness occurs to deteriorate the drawing accuracy. As shown in FIG. 13C, a slender rectangular beam 713 is generated through mutual cutting between apertures 711 and 712. As a pattern is further fined, it is necessary to more accurately adjust the positional relation between the first aperture 711 and the second aperture 712 and it is very difficult to coordinate the apertures 711 and 712 by adjustment. As shown in FIG. 13D, when either aperture finely rotates, for example, the aperture 712 rotates counterclockwise, a formed rectangular beam becomes wedged to cause accuracy deterioration at the oblique-side portion as shown in FIG. 13E.

To form a rectangle (variable rectangle) by an electron beam, there is a method of forming a rectangle by defecting a rectangular beam passing through an upper-stage aperture by a deflecting system of W(width)/H(height) and performing control so that a part of the rectangular beam passes through a lower-stage aperture as shown in FIG. 12.

An oblique portion of a triangle or trapezoid or an oblique wiring pattern is drawn by using the above rectangular beam and thereby forming a fine (slender) beam as shown in FIGS. 13A and 13B. Moreover, there is a method of drawing it by using a triangular aperture and thereby connecting triangles.

Moreover, the official gazette of Japanese Patent Laid-Open No. 238660/1999 discloses a method of drawing a complex figure including a triangular figure and an oblique by preparing a plurality of complex apertures.

There is a first method of drawing an oblique figure, that is, a method of using a slender rectangle as described for the prior art. This method has a problem that an edged portion of an oblique figure becomes stepwise by increasing the height of a rectangle as shown in FIGS. 13A and 13B. Moreover, there is a method of decreasing the height of a rectangle as shown in FIG. 13C. However, this method also has a problem that a rectangle becomes a shape close to a trapezoid as shown in FIG. 13D when first and second apertures rotate from a deflection axis and if drawing the rectangle, the drawing accuracy is deteriorated as shown in FIG. 13E or an electron beam does not resolve a shape when the height is too large. Furthermore, when drawing a shape by a very-thin (small height) rectangular beam, problems occur that the number of figures increases and the throughput deteriorates.

As a second method of drawing an oblique figure, there is a method of using a triangular aperture of the prior art. Because this method performs drawing by using a triangular aperture, the number of figures does not greatly increase compared to the above first method. However, the method has a problem that the joint between figures of a fine pattern causes over-exposure or under-exposure because a 45°-corner portion serves as a joint between figures.

Moreover, the above prior art and the drawing method disclosed in the official gazette of Japanese Patent Laid-Open No. 238660/1999 are effective when drawing an oblique figure for a special purpose. However, it is difficult to apply the above prior art or the method to drawing of a general oblique figure. In the case of a conventional drawing apparatus, however, edge roughness occurs at an oblique portion or it is difficult to accurately draw an oblique portion due to imperfect connection of triangles.

### SUMMARY OF THE INVENTION

The present invention is made to solve the above problems and its object is to provide an electron-beam drawing apparatus and an electron-beam drawing method capable of accurately drawing an oblique figure without deteriorating the throughput in a fine pattern rule.

To achieve the above object, drawing is performed by using a first rectangular aperture in which two opposite sides are parallel with each other and each corner forms a right angle and a second parallelogrammatic aperture in which two opposite sides are parallel with each other and forming a parallelogrammatic electron beam whose sectional size can be changed in accordance with a combination of the first and second aperture.

A first quadrangular aperture in which two opposite sides are parallel with each other and each corner forms a right angle, a second parallelogrammatic aperture in which two opposite sides are parallel with each other, and means for defecting an electron beam passing through the second aperture are used to draw a desired pattern on the surface of a sample.

Moreover, the second parallelogrammatic aperture has an oblique-directional width of 1 μm or less and a longitudinal width that can be changed depending on the first aperture. Oblique-side-portion-contour decomposition means for cutting out the oblique-side portion of a drawn shape at a predetermined with adjusted to the aperture shape is used to draw the cut-out oblique-side portion by using the parallelogrammatic aperture. A code is added to a parallelogram, triangular figure, and a quadrangle of a figure cut out by the oblique-side-portion-contour decomposition means respectively, aperture-number generation means corresponding to the figure codes are used, and the oblique-side portion is drawn by using a variable parallelogrammatic aperture and the inside portion of an oblique side is drawn by using a triangular aperture and a quadrangular aperture.

### BRIEF DESCRIPTION OF THE DRAWINGS

FIG. 1 is an illustration showing a case of forming a beam by a parallelogrammatic aperture;

FIG. 2 is an illustration showing a method for forming a variable parallelogrammatic beam;

FIGS. 3A and 3B are illustrations showing embodiments of parallelogrammatic apertures;

FIG. 4 is an illustration showing triangular apertures for forming variable triangular beams;

FIG. 5 is an illustration showing a configuration of a controller of the present invention;

FIG. 6 is an illustration showing figure shapes and figure codes;

FIGS. 7A and 7B are illustrations showing an embodiment of figure drawing using parallelogrammatic apertures of the present invention;

FIGS. 8A to 8F are illustrations showing a case of drawing a figure including an oblique side in accordance with the conventional method for comparison with the present invention;

FIGS. 9A to 9D are illustrations showing a case of imperfect drawing;

FIGS. 10A to 10C are illustrations showing another embodiment of a drawing method of the present invention;

FIG. 11 is an illustration for supplementing a drawing method of the present invention;

FIG. 12 is an illustration showing a conventional drawing method using a rectangular aperture; and

FIGS. 13A to 13E are illustrations for explaining problems of a conventional rectangular-beam method.

### DETAILED DESCRIPTION OF THE PREFERRED EMBODIMENTS

Embodiments of the present invention are described below.

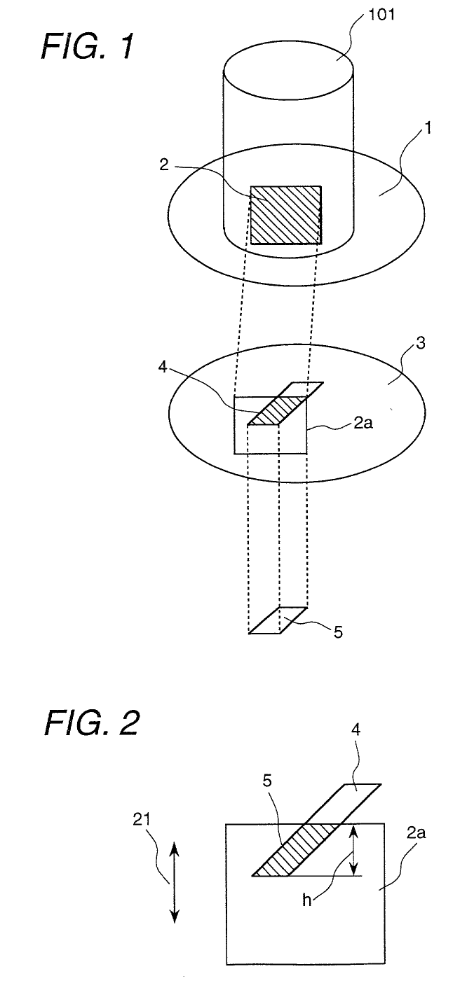

FIG. 1 is an illustration showing an example of beam formation by an electron-beam drawing apparatus of the present invention using a parallelogrammatic aperture. In the case of an electron beam 101, a beam 2a passing through a first aperture 1 having a quadrangular (in which opposite sides are parallel with each other and each corner forms a right angle and which includes an rectangle) opening 2 further passes through a second aperture 3 having a parallelogrammatic opening 4 having two sides parallel with X-axis to generate a parallelogrammatic beam 5. Thus, by combining first and second apertures, a desired beam, in this case, a parallelogrammatic beam is formed.

FIG. 2 is an illustration showing a method for generating a variable parallelogrammatic beam when viewing first and second apertures from an upper portion. The quadrangular beam 2a passing through the quadrangular opening is deflected by a deflecting system and applied onto the parallelogrammatic opening 4 to form the parallelogrammatic beam 5. By controlling the quadrangular beam 2a by the deflecting system in the direction of an arrow 21, the parallelogrammatic beam 5 whose height h can be changed is formed.

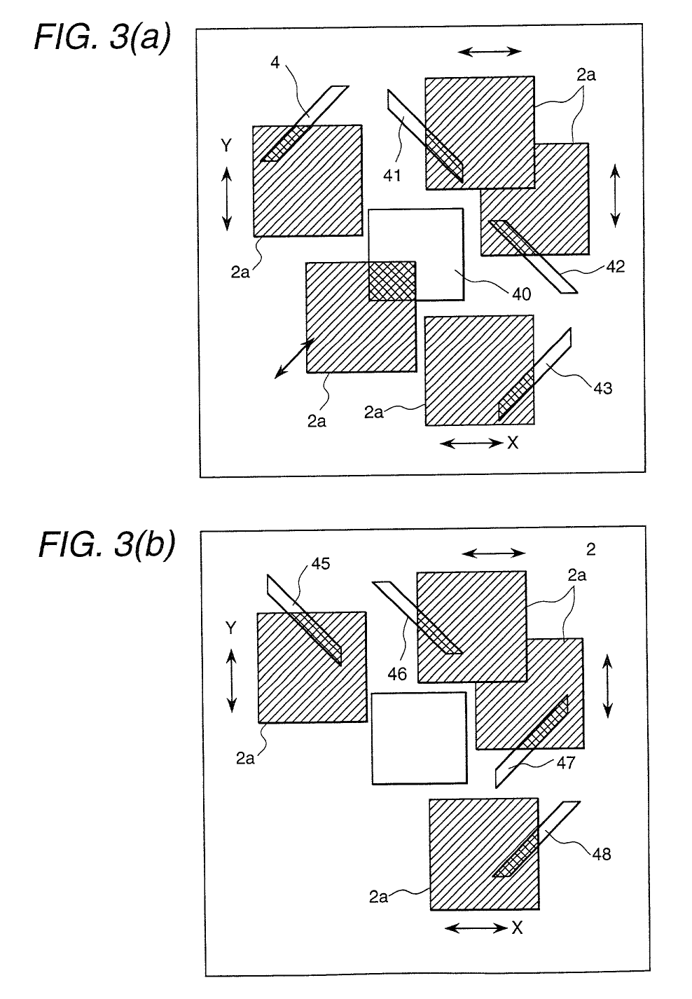

FIGS. 3A and 3B are described below. FIG. 3A is an illustration showing a parallelogrammatic aperture that is constituted of four types of parallelogrammatic openings and quadrangular apertures (including rectangular apertures). In the case of each parallelogrammatic opening, parallelograms 4 and 42 which respectively have two sides parallel with X-axis and whose tilt directions are opposite to each other and parallelograms 41 and 43 which respectively have two sides parallel with Y-axis and whose tilt directions are opposite to each other are formed. Moreover, a case of constituting an example of a quadrangle (rectangle) 40 parallel with XY axes is shown at the central portion. Thus, by changing quadrangular (rectangular) beams 2a formed by the first aperture in the directions shown by arrows, it is possible to variably form the parallelogrammatic beam 5.

FIG. 3B is an illustration showing another embodiment of the parallelogrammatic aperture. It is possible to form an oblique trapezoidal beam by using four types of parallelogrammatic apertures 45, 46, 47, and 48 as shown in FIG. 3B.

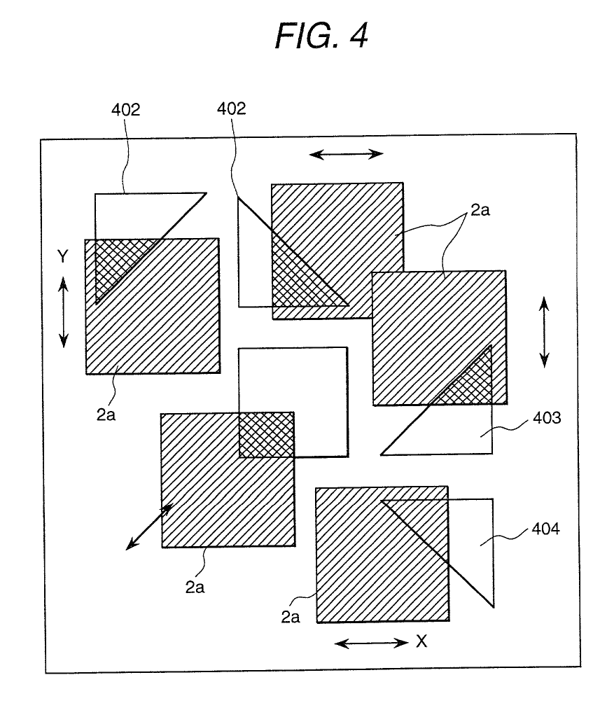

FIG. 4 is an illustration showing triangular apertures (second apertures) for forming variable triangular beams and triangular beams formed by the apertures. The triangular apertures are constituted of four types of triangular openings 401, 402, 403, and 404 which are different from each other in tilt and make it possible to form variable triangular beams by controlling first aperture images, that is, applying positions of beams 2a passing through first apertures in directions of the arrows in FIG. 4 (the central portion in FIG. 4 is used to form a quadrangular beam).

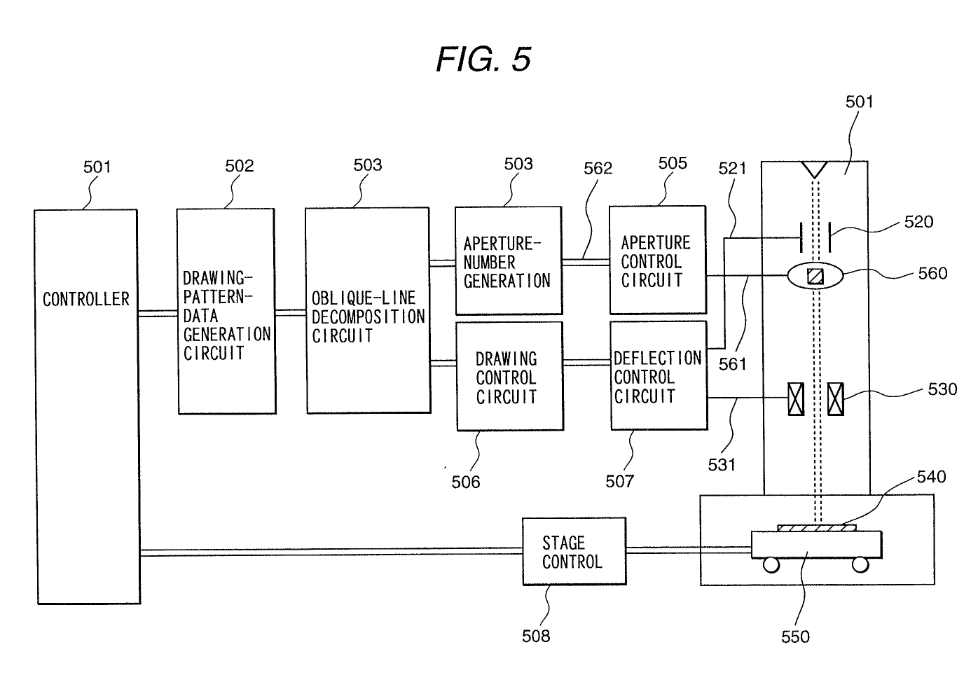

FIG. 5 is an illustration showing a configuration of a controller of the present invention. The whole of an electron-beam drawing apparatus is controlled by a controller 501 and drawing data is transmitted to a drawing-pattern-data generation circuit 502 to control an electron-beam-apparatus body 510 (also referred to as electron-beam enclosure). A stage 550 mounting a wafer 540 is controlled by a state control circuit 508. The drawing data is converted to pattern data using a trapezoid as the basic type by the drawing-pattern-data generation circuit 502 and the oblique-side portion of the trapezoid is decomposed into a contour portion having a constant width and an inside figure excluding a contour oblique side by the oblique-side decomposition circuit 503. Moreover, when the inside figure includes an oblique-side portion, it is decomposed into a triangular figure and a rectangle having sizes that can be drawn. A figure code (signal 562) is added to each figure shape by an aperture-number generation circuit 504 to control an aperture 560 via an aperture control circuit 505 and select and form a beam having a desired shape. The aperture 560 is collectively shown by two apertures constituted of the first aperture 1 and second aperture 3 shown in FIGS. 1 to 4 and a mechanism (electron-beam deflecting system) for applying an electron beam to a predetermined opening (2a). The aperture 560 generates an electron beam having a predetermined shape (parallelogram or triangle) in accordance with an aperture selection signal (electron-beam deflection signal) generated by the aperture control circuit 505 and applies the electron beam onto a wafer.

Moreover, deflection data for positioning a beam having a predetermined figure shape and an exposure-time control signal for turning on/off an electron beam are generated by a drawing control circuit 506, which pass through a deflection control circuit 507, control a deflecting system 530 and a blanking electrode 520 in accordance with a deflection signal 531 and a beam on/off signal 521, and apply an electron beam onto a wafer.

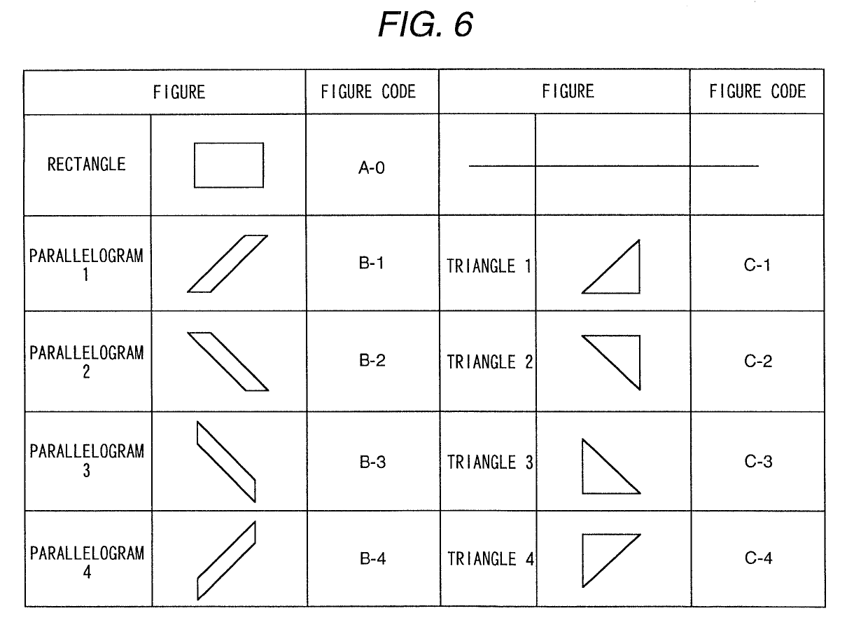

FIG. 6 shows figure shapes and figure codes. The figure codes shown in FIG. 6 are generated for cut-out parallelogram, triangle, and rectangle by the aperture-number generation circuit 504 to select an aperture. Specifically, as figure codes, A-0 is provided to a rectangle, B-1, B-2, B-3, and B-4 are provided to four types of parallelograms one each, and C-1, C-2, C-3, and C-4 are provided to four types of triangles one each.

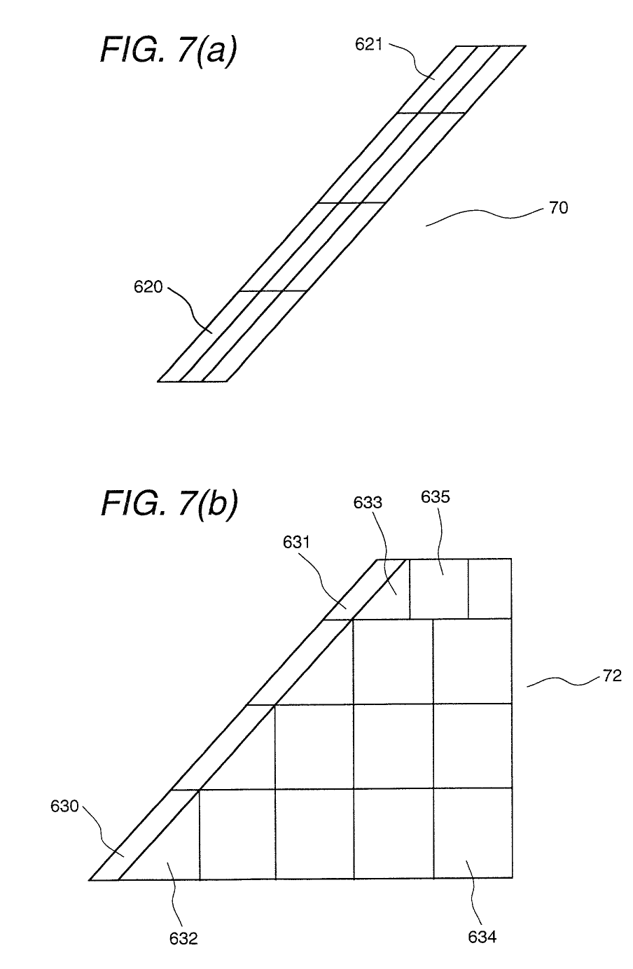

FIGS. 7A and 7B show an embodiment for figure drawing using a parallelogrammatic aperture of the present invention. FIG. 7A shows a thin parallelogram 70 that is drawn by parallelogrammatic beams 620 and 621 whose dimensions are changed in the height direction. FIG. 7B shows an example of cutting out the contour portion of the oblique-side portion of a trapezoid 72 and drawing the contour portion by parallelogrammatic beams 630 and 631. Thereby, the drawing accuracy of the contour portion is improved.

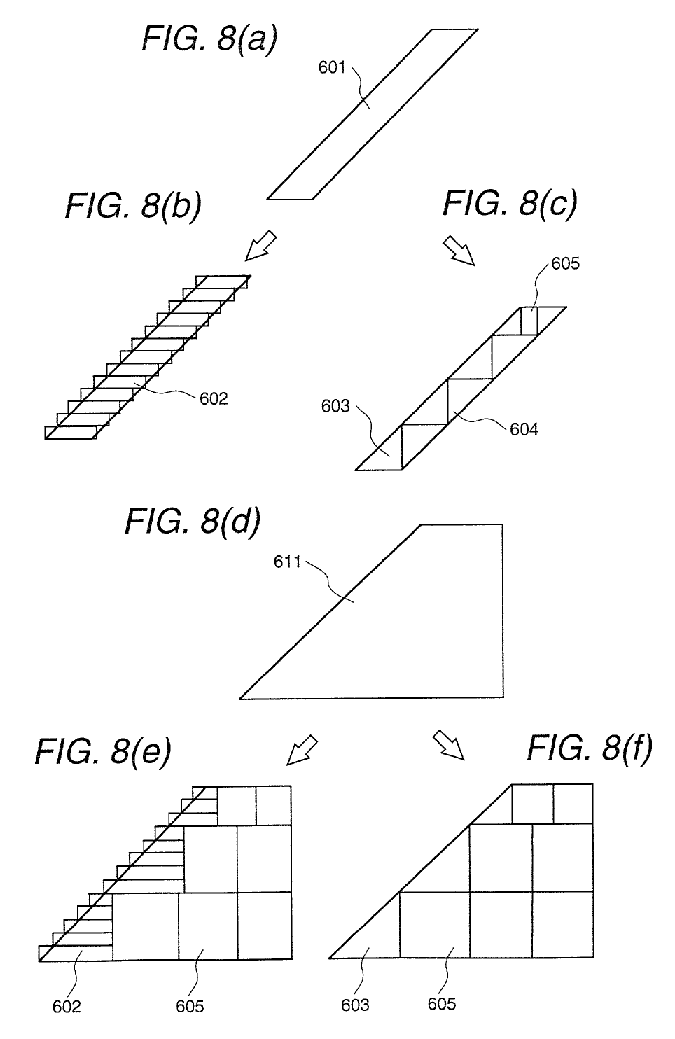

The inside portion of the oblique side is drawn by triangular beams 632 and 633 and quadrangular beams 634 and 635 as shown in FIGS. 8D to 8F. By drawing the portion in accordance with the above method, accuracy deterioration due to under-exposure or over-exposure does not occur at joints between parallelogram 630, triangle 632, and quadrangle (rectangle) 634 even if the accuracy is slightly low.

An example of drawing a figure including an oblique side by a conventional method is shown below for comparison with the present invention.

FIGS. 8A to 8F are illustrations showing a case of drawing a figure including an oblique side by a conventional method. FIGS. 8A to 8C show a case of drawing a shape with a parallelogram 602 having a small height (1 μm or less). A parallelogram 601 to be drawn is drawn by the method in FIG. 8B or 8C. FIG. 8B shows a method an oblique-side portion by using a very-thin rectangular beam 602 having a height of 0.1 μm or less. The method in FIG. 8C performs drawing by using triangular beams 603 and 604. Moreover, drawing is performed by using the triangular beams 603 and 604 and a rectangular beam 605 depending on dimensions of a figure. FIGS. 8D to 8F are illustrations showing a case of drawing a trapezoid. There are two methods of FIGS. 8E and 8F for drawing a trapezoid 611. The method in FIG. 8E draws an oblique-side portion by using the very-thin rectangular beam 602 and the inside portion of the oblique side by using the variable rectangle 605. The method in FIG. 8F draws an oblique-side portion by using the triangular beam 603 formed by a triangular aperture and the inside portion of the oblique side by using the variable rectangle 605. In the case of the method in FIG. 8B or 8C, the accuracy of a contour portion cannot be improved. The same is true for the methods in FIGS. 8E and 8F.

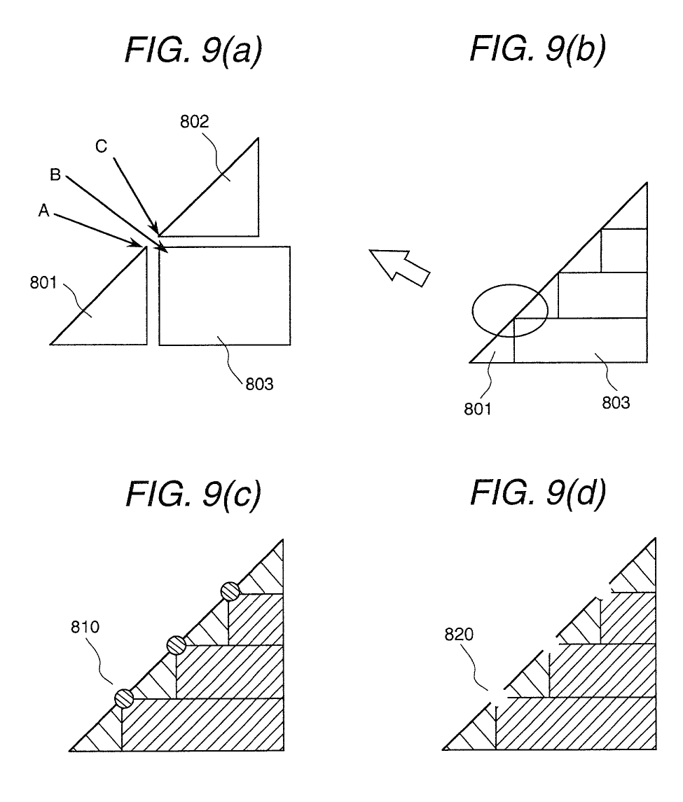

Then, the drawing accuracy of a contour portion is described below by referring to FIGS. 9A to 9D. The method in FIG. 9B is enlarged and shown in FIG. 9A. Because acute-angle corners A and C of an lower triangular FIG. 801 and an upper triangular FIG. 802 are connected with the corner B of a right quadrangular FIG. 803 at one point, it is necessary to coordinate the connection accuracy of a beam formed by each aperture to a very high accuracy. Particularly, it is difficult to secure an accuracy in a fine pattern. Because these three points (A, B, and C) are connected at a contour portion, the drawing accuracy of the contour portion is deteriorated. Moreover, FIGS. 9C and 9D respectively show a drawing result when a problem lies in the connection accuracy of a figure by each aperture. When joints of figures overlap, a connection point 810 causes over-dose (over-exposure) as shown in FIG. 9C and a protrusion is produced at an oblique-side portion. Moreover, when joints between figures separate from each other, under-does (under-exposure) occurs as a connection point 820 shown in FIG. 9D and a hollow is formed at an oblique-side portion. The methods in FIGS. 9C and 9D have the same problem. It is a problem that under-exposure or over-exposure appears at a contour portion.

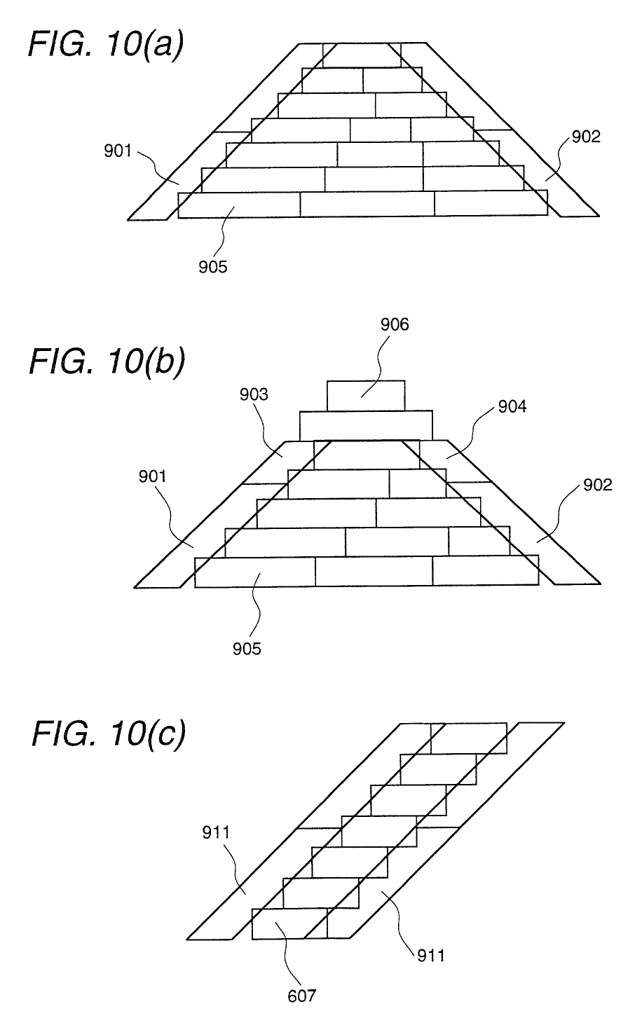

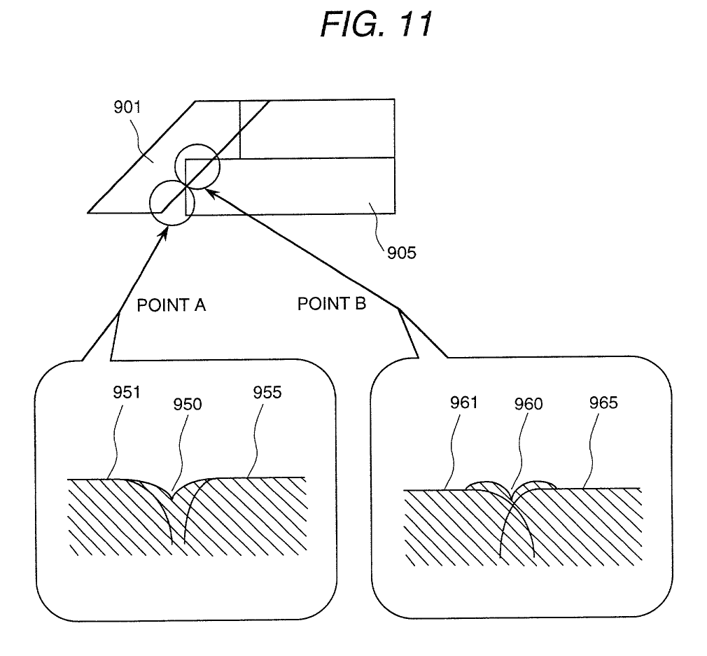

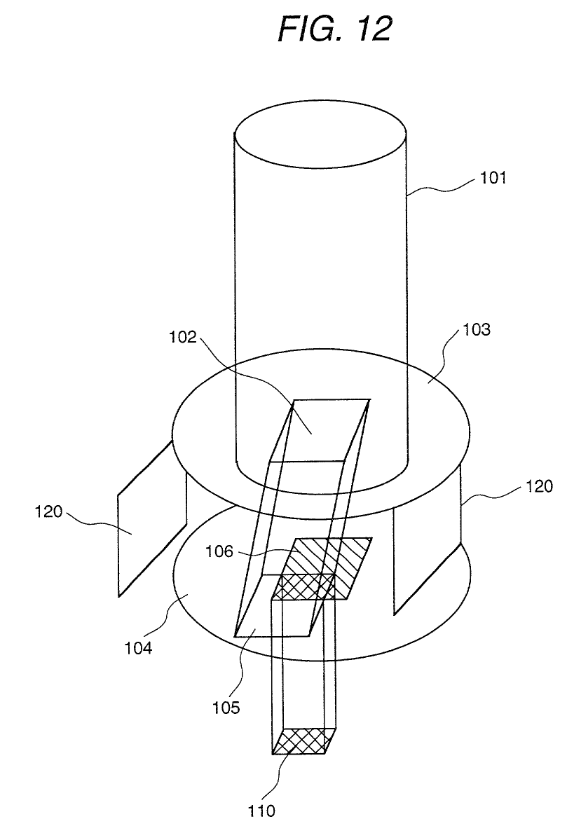

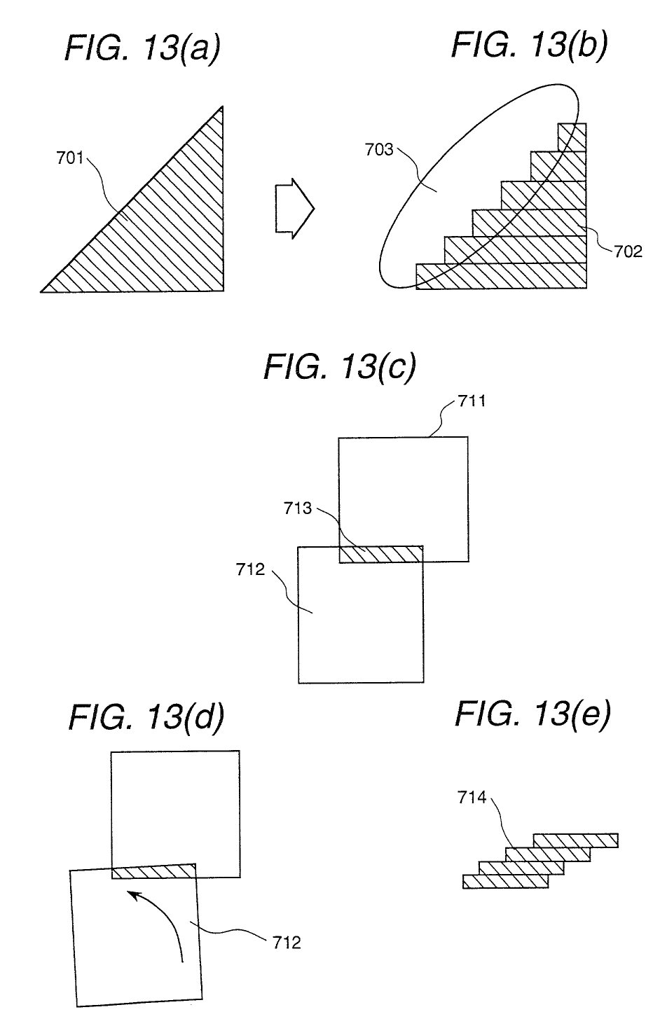

FIGS. 10A, 10B, and 10C show other embodiments of the present invention.

These embodiments are characterized by drawing an oblique-side portion with a parallelogram and drawing an inside figure with a rectangle instead of a triangular figure. FIG. 10A shows an embodiment for drawing a trapezoid, in which an oblique-side portion is drawn by using two types of parallelograms 901 and 902 and an inside figure is drawn by using a rectangle 905. FIG. 9B shows an embodiment for drawing an isosceles triangle, in which an oblique side is drawn by variable-height parallelograms 901, 902, 903, and 904 and the inside is drawn by a rectangle 905. Then, a vertex of a triangle is drawn by using a fine rectangle 906. Moreover, FIG. 9C shows a parallelogram drawing method for drawing a parallelogram by using a parallelogrammatic beam 911 and a rectangular beam 607. By drawing the parallelogram as described above, it is possible to improve the drawing accuracy of a contour portion.

FIG. 11 is an illustration for supplementing the drawing methods in FIGS. 10A to 10C. At the joint between the parallelogram 901 and quadrangle (rectangle) 905, the point A shows a separate state (a gap is produced) and the point B shows an overlapped state. Generally under the above state, under-exposure or over-exposure occurs and the accuracy is deteriorated. However, the under-exposure portion (point A) is exposed (950) larger than actual drawing positions 951 and 955 and the gap is almost absent. Moreover, the point B causes over-exposure because actual drawing positions 961 and 965 overlap each other and protrusions are slightly produced as shown by symbol 960. However, because these under-exposure or over-exposure portion is present inside of a drawn figure, it does not deteriorate the contour-drawing accuracy. Particularly, in the case of a method of cutting out and drawing a contour portion as a method of the present invention, the above phenomenon inside of a contour does not matter at all.

By using the drawing methods shown in FIGS. 10 and 11, it is possible to draw an oblique-side portion without using a very slender rectangle as ever, the throughput is improved. Moreover, because it is unnecessary to use a triangular beam, an advantage is obtained that an aperture is simplified.

According to the present invention, it is possible to accurately draw the contour of a figure without deteriorating the throughput because of drawing a fine oblique pattern by using an oblique parallelogrammatic aperture and a triangular aperture.

What is claimed is:

1. An electron-beam drawing apparatus for drawing a desired pattern by applying an electron beam formed by using a plurality of apertures onto the surface of a sample, comprising a first quadrangular aperture in which two opposite sides are parallel with each other and each corner forms a right angle, a second parallelogrammatic aperture in which two opposite sides are parallel with each other, and deflection means for an electron beam passing through the second aperture, wherein a desired pattern is drawn on the surface of a sample.

2. The electron-beam drawing apparatus according to claim 2, wherein the second parallelogrammatic aperture has a diagonal width of 1 μm or less and a longitudinal width which can be changed depending on the first aperture.

3. The electron-beam drawing apparatus according to claim 1, wherein oblique-side-portion-contour decomposition means for cutting out an oblique-side portion of a drawn shape at a predetermined width adjusted to an aperture shape is included to draw the cut-out oblique-side portion by using the parallelogrammatic aperture.

4. The electron-beam drawing apparatus according to claim 3, wherein a code is added to a parallelogram, a triangle, and a quadrangle of a figure cut out by oblique-side-portion-contour decomposition means respectively and aperture-number generation means corresponding to the figure codes are used to draw an oblique-side portion by using a variable parallelogrammatic aperture and the inside portion of an oblique side by using a triangular aperture and a quadrangular aperture.

5. An electron-beam drawing method for drawing a desired pattern by applying an electron beam formed by using a plurality of apertures onto the surface of a sample, comprising the steps of forming a light beam by a first quadrangular aperture in which two opposite sides are parallel with each other and each corner forms a right angle, forming a light beam by a second parallelogrammatic aperture, deflecting the light beams in accordance with an electron-beam drawn shape passing through the second aperture, and drawing a desired pattern on the surface of a sample.

6. The electron-beam drawing method according to claim 5, wherein an oblique-side contour portion is decomposed, a code is added to a parallelogram, a triangle, and a quadrangle of a figure cut out through decomposition and aperture numbers corresponding to the figure codes are added to draw an oblique-side portion by using a variable parallelogrammatic aperture and the inside portion of an oblique side by using a triangular aperture and a quadrangular aperture.
